(19) United States
(12) Patent Application Publication
(10) Pub. No.: US 2002/0068927 A1
(43) Pub. Date: Jun. 6, 2002

(54) Method and apparatus for myocardial laser treatment

(76) Inventor: Marvin A. Prescott, Los Angeles, CA (US)

Correspondence Address: SHELDON & MAK, INC, 225 SOUTH LAKE AVENUE, 9TH FLOOR, PASADENA, CA, 91101, US

(21) Appl. No.: 09/891,709

(22) Filed: Jun. 26, 2001

Related U.S. Application Data
(60) Provisional application No. 60/214,463, filed on Jun. 27, 2000.

(51) Int. Cl. A61B 18/20

(52) U.S. Cl. 606/12

(57) ABSTRACT

A method and apparatus for myocardial laser treatment.

### CROSS-REFERENCE TO RELATED APPLICATIONS

The present Application claims the benefit of U.S. Provisional Patent Application 60/214,463, filed Jun. 27, 2000, the contents of which are incorporated herein by reference in its entirety.

### BACKGROUND

There remains a need in the art for improved methods and devices for effectively delivering a low power laser treatment to the myocardium or other body organ while monitoring physiological functions and communicating the status of those functions remotely to the physician from the patient's location. There is also a need for a method that would also allow the physician to wirelessly reprogram the patient's treatment device from a site remote to the patient.

Prior art methods and devices do not show a means for monitoring and storing physiological function data while applying a laser treatment with a wearable or implanted device, nor do they show the means to communicate the collected physiological data to the patient's physician or other medical personnel at a remote location. Further, prior art methods and devices do not show the means for allowing the physician to reprogram the patient's device from a remote location after evaluating the patient's physiological status.

Thus, there remains a need for a method and apparatus for performing these functions.

### FIGURES

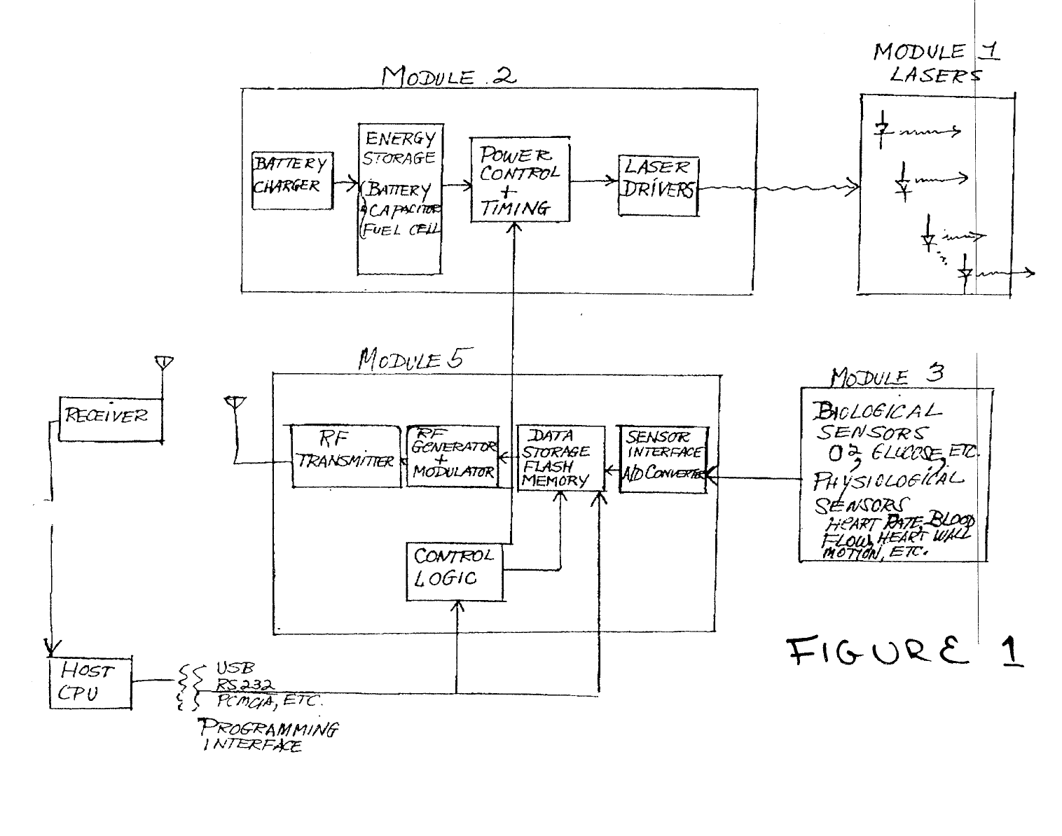

These features, aspects and advantages of the present invention will become better understood with regard to the following description, appended claim and accompanying figures where FIG. 1 is a schematic drawing showing an apparatus according to the present invention for performing a method according to the present invention.

### DESCRIPTION

According to embodiment of the present invention, there is provided a method and apparatus for the laser treatment of myocardium and other tissues. The method and apparatus provide a structural means for monitoring patient physiological functions and biochemical processes, such as heart rate, heart muscle circulation, heart wall motion, blood flow and temperature, and a means for communicating these physiological functions or biochemical processes to a PDA, cell phone, or computer and then remotely to the patient's physician or other medical personnel. Additionally, the modular design of the apparatus, such as using a system on a chip (SOC) for each module, allows the apparatus to be configured in multiple form factors. The apparatus provides a structural means for manufacturing a comfortable, wearable laser treatment device that can be manufactured in various sizes and shapes, thus providing total freedom of design, and solving the problems detailed above.

According to one embodiment, the apparatus for the laser treatment of myocardium comprises a chip scale packaged laser module that can provide low power laser treatment. The apparatus of the present invention further comprises physiological sensors to monitor various body functions such as heart rate, heart blood flow, and heart wall motion, or comprises biochemical sensors to monitor biochemistry such as oxygen saturation or glucose levels, or comprises both types of sensors. The apparatus further comprises a battery module comprising a chip scale packaged battery control/timer that provides battery/power control and timing of the laser activation. The battery module is comfortable but other forms such as button batteries may be used for certain applications.

The apparatus preferably comprises an RF SOC, such as Bluetooth, for wireless connectivity to a PDA, cell phone, computer device, or interact device. The apparatus additionally preferably comprises a means for connecting to the host such as USB, PCMCIA, or RS-232 to transmit physiologic and biochemical data wirelessly to the patients care giver so that laser dosage can adjusted by the care giver from a location remote from the patient.

Referring now to FIG. 1, there is shown a schematic drawing of one embodiment of an apparatus according to the present invention. The present invention provides a laser device which has a modular design and, preferably, has separate systems on a chip (SOC) or modules, where each system of module provides a specific function. As can be seen, Module 1 comprises at least one laser diode chip. Module 1 may be constructed with focusing lenses for certain applications or without focusing lenses for other applications, as will be understood by those with skill in the art.

Module 2 comprises an SOC which is an ASIC that has a programmable logic IC's to control the power timing and output of the laser beam treatment, and an ASIC to control battery power utilization and recharge.

Module 3 comprises physiological sensors or biochemical sensors or both, such as photo sensors, photoacoustic sensors or miniature fiberoptic sensors which monitor body functions such as heart rate, heart wall motion, temperature and blood flow, or sensors to measure oxygen content of the blood or other biochemical molecules.

Module 5 comprises a SOC, such as Bluetooth, for RF transmission wirelessly to the patient's computer, PDA, wireless phone or other portable wireless device. The patient's physician can then access the data and reprogram the apparatus by transmitting the new program to the device. Security software is preferably included in the software to prevent unauthorized access to the data. Module 5 additionally preferably comprises a sensor interface with the DSP Module for receiving physiological data from Module 3, and for transferring the data to a data storage chip with embedded software for transmission to the RF generator and, then, to the RF transmitter. A control logic controls the functions of Module 2 and 5.

Additionally, a pholodiode sensor chip (not shown) can be implanted under the patient's skin in order to guide the laser treatment beams to the treatment area(s), such as the heart and or coronary arteries, and/or to monitor heart function or monitor biochemical processes within the heart and body and transmit these to the sensor interface in Module 5.

Although the present invention has been discussed in considerable detail with reference to certain preferred embodiments, other embodiments are possible. Therefore, the scope of the appended claims should not be limited to the description of preferred embodiments contained in this disclosure.

I claim:

1. A method and apparatus for myocardial laser treatment as disclosed herein.
